(19) United States
(12) Patent Application Publication
(10) Pub. No.: US 2024/0150272 A1
(43) Pub. Date: May 9, 2024

(54) PROCESSES FOR PURIFYING GLYCOL ETHERS

(71) Applicant: Dow Global Technologies LLC, Midland, MI (US)

(72) Inventors: Qi Jiang, Shanghai (CN); Kaoru Ohba, Tokyo (JP); Yujun Liu, Pearland, TX (US)

(21) Appl. No.: 18/548,990

(22) PCT Filed: May 19, 2021
(86) PCT No.: PCT/CN2021/094703

§ 371 Date: Sep. 5, 2023

(51) Int. Cl. C07C 41/42

(52) U.S. Cl. CPC C07C 41/42

(57) ABSTRACT

A processes for purifying glycol ethers, comprises (a) providing a glycol ether to a first vessel, the glycol ether, the glycol ether having the following formula: R₁—O— (CHR₂CHR₃) O)ₙR₄; wherein R₁ is an alkyl group having 1 to 9 carbon atoms or a phenyl group; wherein R₂ and R₃ each individually is hydrogen, a methyl group or an ethyl group, provided that when R₃ is a methyl group or an ethyl group, R₂ is hydrogen and provided that when R₂ is a methyl group or an ethyl group, R₃ is hydrogen; wherein R₄ is hydrogen, an alkyl group having 1 to 4 carbon atoms, an acetyl group, or a propionyl group; and wherein n is an integer of 1 to 3; (b) filling the first vessel with inert gas; (c) heating the glycol ether in the first vessel to a sub-boiling temperature, wherein the sub-boiling temperature is at least 15° C. less than the normal boiling point of the glycol ether; (d) cooling the vapor from the first vessel in a second vessel to provide a liquid; and (e) contacting the glycol ether with a mixed bed of ion exchange resins comprising cationic exchange resins and anionic ion exchange resins.

### FIELD

The present invention relates to processes for purifying glycol ethers by removing metal contaminants and other impurities.

### INTRODUCTION

A pure solvent, that is, a solvent which is free of ionic contaminants, is typically required for many industrial purposes such as for the manufacture of pharmaceuticals and electronic materials. For example, organic solvents with a very low level of metallic ion contaminants are required for semiconductor fabrication processes, because metallic ion contaminants negatively affect the performance and production yield of the fabricated semiconductor devices. Some hydrophilic organic solvents, such as propylene glycol methyl ether (PGME), and hydrolysable solvents, such as propylene glycol methyl ether acetate (PGMEA), are commonly used for lithography processes in semiconductor fabrication processes. And, when those organic solvents are to be used in semiconductor fabrication processes, it is desired that such solvents have a very low level (e.g., less than 50 parts per trillion [ppt] in some cases) of metallic ion contaminants.

Heretofore, some ion exchange resins have been used for purifying various organic solvents by removing metallic ionic contaminants from the organic solvents. And, the purification of organic solvents using ion exchange technology has been applied to organic solvents which are used in manufacturing electronic materials. For example, references that disclose a process for purifying an organic solvent using an ion exchange resin include JP1989228560B; JP2009057286A; JP5,096,907B; and U.S. Pat. Nos. 7,329,354; 6,123,850; and 5,518,628.

Distillation is another technique that has been used to purify chemicals to achieve electronic grade purities.

However, such prior approaches can be complicated and difficult to implement. It would be desirable to have new processes for purifying glycol ethers that achieve a desired level of purity and that are easy to implement.

### SUMMARY

The present invention is directed to processes for purifying glycol ethers. In various embodiments, the present invention can purify the glycol ethers to very low levels of metallic ions and other contaminants. In some embodiments, the present invention advantageously provides processes for purification of glycol ethers that are easier to implement than prior approaches.

In one embodiment, a process for purifying glycol ethers comprises (a) providing a glycol ether to a first vessel, the glycol ether having a normal boiling point at one bar and the glycol ether having the following formula:

R₁—O—(CHR₂CHR₃)O)ₙR₄
wherein R₁ is an alkyl group having 1 to 9 carbon atoms or a phenyl group; wherein R₂ and R₃ each individually is hydrogen, a methyl group or an ethyl group, provided that when R₃ is a methyl group or an ethyl group, R₂ is hydrogen and provided that when R₂ is a methyl group or an ethyl group, R₃ is hydrogen; wherein R₄ is hydrogen, an alkyl group having 1 to 4 carbon atoms, an acetyl group, or a propionyl group; and wherein n is an integer of 1 to 3; (b) filling the first vessel with inert gas; (c) heating the glycol ether in the first vessel to a sub-boiling temperature, wherein the sub-boiling temperature is at least 15° C. less than the normal boiling point; (d) cooling the vapor from the first vessel in a second vessel to provide a liquid; and (e) contacting the glycol ether with a mixed bed of ion exchange resins comprising cationic ion exchange resins and anionic ion exchange resins.

Various embodiments of the present invention are described in more detail in the following Detailed Description.

### BRIEF DESCRIPTION OF FIGURE

FIG. 1 is a flow diagram illustrating a process for purifying glycol ethers according to one embodiment of the present invention.

### DETAILED DESCRIPTION

As used throughout this specification, the abbreviations given below have the following meanings, unless the context clearly indicates otherwise: BV/hr=bed volume/hour(s), μm=micron(s), nm=nanometer(s), g=gram(s); mg=milligram(s); L=liter(s); mL=milliliter(s); ppm=parts per million; ppb=parts per billion; ppt=parts per trillion; m=meter(s); mm=millimeter(s); cm=centimeter(s); min=minute(s); s=second(s); hr=hour(s); ° C.=degree(s) Celsius; %=percent, vol %=volume percent; and wt %=weight percent.

In general, the present invention relates to processes for purifying glycol ethers. The glycol ethers that can be purified using such processes include glycol ether acetates and have the following formula:

R₁—O—(CHR₂CHR₃)O)ₙR₄
wherein R₁ is an alkyl group having 1 to 9 carbon atoms or a phenyl group; wherein R₂ and R₃ each individually is hydrogen, a methyl group or an ethyl group, provided that when R₃ is a methyl group or an ethyl group, R₂ is hydrogen and provided that when R₂ is a methyl group or an ethyl group, R₃ is hydrogen; wherein R₄ is hydrogen, an alkyl group having 1 to 4 carbon atoms, an acetyl group, or a propionyl group; and wherein n is an integer of 1 to 3. Examples of glycol ethers that can be purified according to various embodiments of the present invention include propylene glycol methyl ether, dipropylene glycol methyl ether, tripropylene glycol methyl ether, propylene glycol ethyl ether, propylene glycol propyl ether, ethylene glycol propyl ether, ethylene glycol butyl ether, diethylene glycol methyl ether, diethylene glycol ethyl ether, diethylene glycol propyl ether, diethylene glycol butyl ether, propylene glycol methyl ether acetate, dipropylene glycol methyl ether acetate, dipropylene glycol methyl ether diacetate, and mixtures thereof.

Among the properties important in characterizing the glycol ethers for use in the inventive processes is the normal boiling point. As used herein, the “normal boiling point” is the boiling point of the glycol ether measured at one bar.

In one aspect, a process for purifying glycol ethers (as described herein) comprises (a) providing a glycol ether to a first vessel; (b) filling the first vessel with inert gas; (c) heating the glycol ether in the first vessel to a sub-boiling temperature, wherein the sub-boiling temperature is at least 15° C. less than the normal boiling point of the glycol ether; (d) cooling the vapor from the first vessel in a second vessel to provide a liquid; and (e) contacting the glycol ether with a mixed bed of ion exchange resins comprising cationic ion exchange resins and anionic ion exchange resins. In some embodiments, steps (c) and (d) are performed before step (e), with the glycol ether in step (e) being the liquid from step (d). In other words, in such embodiments, the sub-boiling separation is performed before ion exchange. In other embodiments, step (e) is performed before steps (a)-(d), wherein the glycol ether exiting the mixed bed of ion exchange resins is provided to the first vessel in step (a). In other words, in such embodiments, the ion exchange step occurs prior to the sub-boiling separation.

In some embodiments, after the process steps are completed, the concentration of Li, Na, Mg, K, Ca, Al, Fe, Ni, Zn, Cu, Cr, Mn, Co, Sr, Ag, Cd, Cs, Ba, Pb, and Sn in the glycol ether are each 1 ppb or less. In some embodiments, after the process steps are completed, the concentration of Li, Na, Mg, K, Ca, Al, Fe, Ni, Zn, Cu, Cr, Mn, Co, Sr, Ag, Cd, Cs, Ba, Pb, and Sn in the glycol ether are each 100 ppt or less.

In some embodiments, the water content and the oxygen content in the first vessel are each less than 20 ppm.

In some embodiments, the cationic ion exchange resins are weak-acid cationic ion exchange resins, and the anionic ion exchange resins are weak-base anionic ion exchange resins. In some further embodiments, the weak-acid cationic ion exchange resin is a macroreticular type resin and the weak-base anionic ion exchange resin is a macroreticular type resin. In some further embodiments, the matrix material of the macroreticular type resins are selected from the group consisting of a crosslinked styrene-divinyl benzene copolymer, a crosslinked acrylic (methacrylic) acid-divinyl benzene copolymer, or a mixture thereof. In some further embodiments, the weak-acid functionality of the weak-acid cationic ion exchange resins is selected from the group consisting of weak-acid carboxylic acid groups, weak-acid phosphonic acid groups, weak-acid phenolic groups, and mixtures thereof. In some further embodiments, the weak-base functionality of the weak-base anionic ion exchange resins is selected from the group consisting of a primary amine group, a secondary amine group, a tertiary amine group, and mixtures thereof.

In some embodiments, the cationic ion exchange resins are strong-acid cationic ion exchange resins, and the anionic ion exchange resins are strong-base anionic ion exchange resins.

Processes of the present invention include a sub-boiling step. The sub-boiling step involves heating the glycol ether to a temperature that is at least 15° C. lower than the normal boiling point of the glycol ether. First, the glycol ether is provided to a first vessel. The first vessel is then filled with an inert gas such as nitrogen or argon. The purity of the inert gas is at least 99.999%. As the inert gas flows into the first vessel, it should pass through a gas filter to remove particles and dust in order to maintain the purity of the gas. In addition, the water content and oxygen content are controlled to less than 20 ppm using techniques known to those having ordinary skill in the art based on the teachings herein. The contents of the first vessel are then heated to a temperature that does not exceed a sub-boiling temperature that is 15° C. less than the normal boiling point of the glycol ether. The heating of the glycol ether in the first vessel generates a vapor. The vapor flows out of the first vessel through a pipe or other conduit into a second vessel. In the second vessel, the vapor is allowed to cool naturally and condense into a liquid. For the glycol ethers contemplated herein, the temperature of the liquid in the second vessel should be kept no higher than 20° C. Thus, the sub-boiling procedure, in some embodiments, comprises (a) providing the glycol ether to a first vessel; (b) filling the first vessel with inert gas; (c) heating the glycol ether in the first vessel to a sub-boiling temperature, wherein the sub-boiling temperature is at least 15° C. less than the normal boiling point of the glycol ether; and (d) cooling the vapor from the first vessel in a second vessel to provide a liquid.

If ion exchange has previously been performed on the glycol ether prior to sub-boiling, the purified glycol ether may be collected for use. If the glycol ether has not passed through the ion exchange procedure, the glycol ether from the second vessel in the sub-boiling step may proceed to the ion exchange procedure as described further herein.

The ion exchange portion of the present invention includes the use of a mixed bed of ion exchange resin. A mixed bed of ion exchange resin refers to a mixture of at least: (1) a cationic ion exchange resin and (2) an anionic ion exchange resin. In some embodiments, the cationic ion exchange resin used in the mixed bed of ion exchange resin is a weak-acid cationic ion exchange resin, and the anionic ion exchange resin used in the mixed bed of ion exchange resin is a weak-base anionic ion exchange resin. In some embodiments, the cationic ion exchange resin used in the mixed bed of ion exchange resin is a strong-acid cationic ion exchange resin, and the anionic ion exchange resin used in the mixed bed of ion exchange resin is a strong-base anionic ion exchange resin. In some embodiments where the glycol ether is a hydrolysable glycol ether ester, the cationic ion exchange resin used in the mixed bed of ion exchange resin is a strong-acid cationic ion exchange resin, and the anionic ion exchange resin used in the mixed bed of ion exchange resin is a weak-base anionic ion exchange resin, although weak-acid cationic ion exchange resin in combination with weak-base anionic ion exchange resin can also be used in other embodiments.

It is commonly known that the degree of swelling of a gel-type resin is dependent on a solubility parameter of solvents; and that a macroreticular (MR)-type resin is dimensionally stable in an organic solvent, for example, as described in “Behavior of Ion Exchange Resins in Solvents Other Than Water—Swelling and Exchange Characteristics”, George W. Bodamer, and Robert Kunin, Ind. Eng. Chem., 1953, 45 (11), pp 2577-2580. In one preferred embodiment, an ion exchange resin useful in the present invention that exhibits “dimensional stability” refers to an ion exchange resin wherein the volume of the soaked ion exchange resin in an organic solvent changes less than +10 percent compared to the volume change of the soaked resin in water (i.e., a hydrated resin).

Not to be limited to any particular theory, in the case of a gel-type resin, metal ions are assumed to be trapped on the surface of the ion exchange beads first, and then the metal ions are assumed to diffuse into the inside of polymer beads. Ion exchange capacity known by those skilled in the art from product technical sheets for ion exchange resins are expressed in chemical equivalent/unit volume regardless of where ion exchange sites are located in the resin beads. When ion exchange capacity can be fully utilized, metal removal capability and capacity are maximized. Solvent absorbed in the resin beads carries metal ions into the inside of the resin bead. If the ion exchange resin bead does not absorb solvent and resin molecules are tightly packed, metal ions cannot migrate into inside of polymer beads. The degree of resin swelling indicates how much solvent is absorbed. Since gel type ion exchange resins are designed to contain water at 40% to approximately (˜) 60% of the hydrated resin bead (i.e., ion exchange resins inherently have a strong affinity to water or water miscible solvents), swelling of the ion exchange resin will become less obvious as hydrophobicity of solvents increased, for example, as the ratio of hydrophilic solvent of the mixed resin is decreased. When there is a lack of presence of solvent in the resin beads, ion exchange sites located in inside of the resin beads cannot be utilized in ion exchange reactions. This results in a degradation of metal removal efficiency and metal removal capacity. In an extreme case, only ion exchange sites located on the resin beads' surface are active in contact with a hydrophobic solvent.

For the case of an MR-type resin, the resin has more surface area because of macro-pores located on the bead surface; the principle being that ion exchange reactions takes place mainly at the pores located on the resin bead surface. Also, to prevent corruption of the macro-pore structures of the resins, the resins are designed to stabilize the dimension and the surface morphology of resin beads. A benefit of using an MR-type resin is that even a hydrophobic solvent has a minimal deleterious impact on the size and surface morphology of the ion exchange resin; and as a result, the number of ion exchange sites that can be utilized for metal removal are not changed by hydrophobicity of solvent, in other words, by ratio of the hydrophilic solvent and the hydrolysable solvent in mix solvent.

Weak-Acid Cationic Ion Exchange Resin and Weak-Base Anionic Ion Exchange Resin

In some embodiments, the cationic ion exchange resin used in the mixed bed of ion exchange resin is a weak-acid cationic ion exchange resin, and the anionic ion exchange resin used in the mixed bed of ion exchange resin is a weak-base anionic ion exchange resin. An MR-type ion exchange resin is used for the weak-acid cationic ion exchange resin and for the weak-base anionic ion exchange resin used in the mixed resin bed of some embodiments of the present invention. The matrix material of the MR-type resins can be selected from a cross linked styrene—divinyl benzene copolymer (styrene-DVB), an acrylic (methacrylic) acid-divinyl benzene copolymer; or mixtures thereof.

The weak-acid cationic ion exchange resin useful in some embodiments of the present invention includes, for example, a cationic ion exchange resin with at least one kind of weak-acid functionality such as weak-acid carboxylic acid groups, weak-acid phosphoric acid groups, weak-acid phenolic groups, and mixtures thereof. As used herein, such groups are called “weak-acid group(s)”.

Exemplary of some of the commercial weak-acid cationic ion exchange resins useful in the present invention include, for example, AMBERLITE™ IRC76 and DOWEX™ MAC-3 (both of which are available from Dupont); and mixtures thereof.

The weak-base anionic ion exchange resin useful in the present invention includes, for example, an anionic ion exchange resin with at least one kind of weak-base functionality such as primary, secondary or tertiary amine (typically, dimethyl amine) groups, or mixtures thereof. As used herein, such groups are called “weak-base group(s)”.

Exemplary of some of the commercial weak-base anionic ion exchange resins useful in the present invention include, for example AMBERLITE™ 1RA98, AMBERLITE™ 96SB, and AMBERLITE™XE583 as examples of a MR-type styrene polymer matrix; and AMBERLITE™ IRA67 as an example of a gel-type acrylic polymer matrix (all of which are available from Dupont); and mixtures thereof.

In one preferred embodiment, using a weak-acid cationic ion exchange resin in the mixed resin bed of the present invention can minimize the organic impurities generated from side-reactions of ion exchange.

Weak-acid cationic ion exchange resin groups, in general, have a lower affinity to metal cationic ions than strong-acid cationic ion exchange resin groups. It has been found that the metal removal efficiency of the weak-acid cationic ion exchange resin groups is lower than the strong-acid cationic ion exchange resin groups when the weak-acid cationic ion exchange resin is used as single bed. Also, it has been found that by mixing the weak-acid cationic ion exchange resin with the weak-base anionic ion exchange resin, an excellent metal removal capability from both hydrophilic solvent and hydrolysable solvent can be achieved.

One of the benefits of using a mixed resin bed of cation exchange resin and anion exchange resin is that such mixed resin bed provides a higher capability of removing metal from solvent than a single cation exchange resin bed. The mechanism of metal ion removal is a cation exchange reaction. When a metal ion is absorbed in a cation exchange resin, a proton is released. Since the ion exchange reaction is an equilibrium reaction, by removing a proton from the reaction system, a high efficiency of metal ion removal can be achieved. Also, the free proton can cause various side-reactions. In the mixed resin bed, the proton is neutralized and removed from the reaction system thanks to the effect of the anion exchange resin. Counter anions are typically present together with metal cations. For the case of strong base anion exchange resin, the anion exchange resin can absorb the counter anion and release hydroxyl ions, and the protons released from cation exchange reaction react with the hydroxyl ions released from anion exchange reaction, and form water molecules. However, water can be fuel for hydrolysis reaction if the water is added to a hydrolysable solvent.

An advantage of using a mix resin formulation containing a weak-base anionic ion exchange resin includes, for example, such mixed resin bed minimizes the hydrolysis decomposition of hydrolysable solvent. When a solvent to be purified is contacted with a cationic ion exchange resin, protons are released as usual, and the released protons associate with unshared electron pairs of the nitrogen atoms within the weak-base group. By absorbing protons, the weak-base group has a positive electron charge. Then an anionic impurity is bound to the weak-base group due to the charge neutral requirement. Consequently, undesired components such as water are not generated by the purification process of the present invention. Thus, using the weak-base anionic ion exchange resin in a mixed bed of ion exchange resin provides purification of hydrolysable organic solvents without undesirable hydrolysis.

An advantage of using a mix resin formulation containing a weak-acid cationic ion exchange resin includes, for example, such mixed resin bed minimizes the risk of hydrolysis decomposition which can be caused by cationic ion exchange resin localization. Partial localization of cationic ion exchange resin can happen when the uniformity of the mixture in the resin bed collapses during the resin bed construction process due to the difference of sedimentation velocity of ion exchange resins. Localization of cation exchange resin can increase the risk of side-reactions such as hydrolysis during the purification of the solvents, because proton released from cation exchange reaction is active until being neutralized, and the generated impurities are not reversible even after protons are deactivated. Weak-acid cationic ion exchange resin can reduce the risk of hydrolysis, even if localization happens.

The distribution of bead size of the weak-acid cationic ion exchange resin and the weak-base anionic ion exchange resin includes, for example, a bead size of from 100 μm to 2,000 μm in one embodiment, from 200 μm to 1,000 μm, in another embodiment, and from 400 μm to 700 μm in still another embodiment. In one embodiment, the pore size of an MR-type ion exchange resin beads includes, for example, a pore size of from 1 nm to 2,000 nm. In the case of a gel-type resin, the pore size of the beads includes, for example, a pore size of from 0.01 angstroms to 20 angstroms in one embodiment.

The blend ratio of an ion exchange resin combination of a MR-type weak-acid cationic ion exchange resin and an MR-type weak-base anionic ion exchange resin includes, for example, a blend ratio of from 1:9 to 9:1 in volume (or in chemical equivalency) in one embodiment; and from 3:7 to 7:3 in another embodiment. In a preferred embodiment, the blend ratio of the cation exchange resin:anion exchange resin is 5:5. If a blend ratio of cation:anion exchange resin above 9:1 is used or if a blend ratio of cation:anion exchange resin below 1:9 is used, the metal removal rate will be depressed significantly.

Strong-Acid Cationic Ion Exchange Resin and Strong-Base Anionic Ion Exchange Resin

In some embodiments, the mixed bed of ion exchange resins comprises a strong-acid cationic ion exchange resin and a strong-base anionic ion exchange resin. Mixed beds utilizing a strong-acid cationic ion exchange resin and a strong-base anionic ion exchange resin can be useful for glycol ethers other than glycol ether acetates as some combinations of strong-acid cationic ion exchange resins and strong-base anionic ion exchange resins can cause hydrolysis of glycol ether acetates.

In such embodiments, the strong acid cationic ion exchange resins are hydrogen (H)-form strong-acid cationic ion exchange resins, which include cation exchange groups attached to polymer molecules which form resin beads. Examples of such H-form strong acid cation exchange groups include sulfonic acids. A H-form strong acid cation exchange group, such as a sulfonic acid, easily releases a proton (Hi) in exchange with a cationic impurity in the hydrophilic organic solvent. The resin beads of the cationic exchange resins are a polymer with normally spherical shape formed from a composition comprising styrene and divinylbenzene. Thus, in some embodiments, a H-form strong acid cationic exchange resin comprises sulfonic acid attached to polymer molecules formed from a composition comprising styrene and divinylbenzene.

The moisture holding capacity of such strong-acid cationic ion exchange resins used in the mixed bed of ion exchange resins is from 40 to 55 wt %. The moisture holding capacity refers an amount of water in an ion exchange resin when the ion exchange resin is in a hydrated state (swelled in water). The moisture holding capacity varies with many factors, principally a chemical structure of the base resin (styrene-type or acrylic-type), degrees of crosslink of base resin, morphological type of base resin bead (gel-type or MR-type) and, size of ion exchange resin beads, population of cation exchange groups. In some preferred embodiments, the moisture holding capacity of the strong-acid cationic ion exchange resins is from 45 to 50 wt % in a hydrated state. As used herein, the moisture holding capacity is calculated by the following method: a content of water in the strong-acid cationic ion exchange resins is calculated by comparison of the weights of ion exchange resin before and after drying. The drying condition is at 105° C. for 15 hours under a 20 mmHg vacuum, followed by cooling in desiccators for 2 hours. The degreased weight after drying based on a hydrated state ion exchange resin is used to determine the moisture holding capacity based on the following formula:

Moisture Holding Capacity=(Weight of Hydrated Ion Exchange Resin−Weight of Ion Exchange Resin after Drying)*100/Weight of Hydrated Ion Exchange Resin

Regarding the strong-base anionic ion exchange resin used in the mixed bed of ion exchange resins in such embodiments, the strong-base anionic ion exchange resin has anion exchange groups attached to resin beads. In some embodiments, the strong-base anionic ion exchange resin comprises trimethyl ammonium groups (called Type I) or dimethyl ethanol ammonium groups (Type II) attached to polymer molecules which form anionic ion exchange resin beads. The strong-base anionic ion exchange resin releases hydroxyl ions (OH⁻) in exchange with anionic contaminants in a hydrophilic organic solvent. The resin beads of the anionic ion exchange resins are also a polymer with normally spherical shape formed from a composition comprising styrene and divinylbenzene. Thus, in some embodiments, a strong base anionic exchange resin comprises trimethyl ammonium and/or dimethyl ethanol ammonium groups on a resin bead formed from a composition comprising styrene and divinylbenzene. Although the moisture holding capacity of the strong-base anionic ion exchange resins are not particularly limited, the moisture holding capacity is from 55 to 65 wt % when measured as described above, in some preferred embodiments.

In some embodiments where the mixed bed comprises strong-acid cationic ion exchange resins and strong-base anionic ion exchange resins, the resins are gel-type resins. As used herein, and as generally understood in the field of ion exchange resins, a gel-type resin refers to a resin that has a very low porosity (less than 0.1 cm³/g), a small average pore size (less than 1.7 nm) and a low B.E.T. surface area (less than 10 m²/g). Porosity, average pore size and B.E.T. surface area can be measured by the nitrogen adsorption method shown in ISO 15901-2. Such ion exchange resins are distinct from macroporous-type ion exchange resins having a macro-reticular structure (MR-type ion exchange resin) and a macro pore size that is clearly larger than the porosity of gel-type ion exchange resins.

The ratio of strong-acid cationic ion exchange resin to strong-base anionic ion exchange resin when used the mixed bed of ion exchange resins is generally from 1:9 to 9:1 in equivalence ratio of ion exchange groups, in some embodiments. Preferably, the ratio is from 2:8 to 8:2.

Strong-Acid Cationic Ion Exchange Resin and Weak-Base Anionic Ion Exchange Resin

In some embodiments, the mixed bed of ion exchange resins comprises a strong-acid cationic ion exchange resin and a weak-base anionic ion exchange resin. Mixed beds utilizing a strong-acid cationic ion exchange resin and a weak-base anionic ion exchange resin can be useful for hydrolysable glycol ether esters such as propylene glycol methyl ether acetate, dipropylene glycol methyl ether acetate, and dipropylene glycol methyl ether diacetate. In such embodiments, the strong-acid cationic ion exchange resin and the weak-base anionic exchange resin can be any of those disclosed herein.

“Purity loss” is measured by conventional methods such as by gas chromatography-flame ionization detector (GC-FID); and the color property of the solvent is not adversely impacted by the ion exchange process, i.e., the color of the solvent does not increase by using the ion exchange resins of the present invention. “Color” is measured, for example, by using a Pt—Co colorimeter and the method described in ASTM D5386.

When contacting a glycol ether with a mixed bed of ion exchange resin, any known conventional methods for contacting liquids with ion exchange resins can be used. For example, a mixed bed of ion exchange resin can be packed in a column and the glycol ether can be poured from the top of the column through the mixed bed of ion exchange resin. In the contacting step (b) of the process, the flow rate of the glycol ether passing through the mixed resin bed can be, for example, from 1 BV/hr to 100 BV/hr in one embodiment and from 1 BV/hr to 50 BV/hr in another embodiment. If the flow rate of the glycol ether passing through the mixed resin bed is above 100 BV/hr, the metal removal rate will decrease; and if the flow rate of the glycol ether passing through the mixed resin bed is below 1 BV/hr, the purification productivity will decrease; otherwise, a large resin bed will be required to achieve target production throughput. As used herein, “BV” means bed volume, and refers to an amount of liquid contacted with the same amount of a hydrated wet mixed bed of ion exchange resin. For example, if 120 mL of a hydrated wet mixed bed of ion exchange resin is used, 1 BV means 120 mL of glycol ether is contacted with the mixed bed of ion exchange resin. “BV/hr” is calculated by flow rate (mL/hr) divided by bed volume (mL).

In general, the temperature of the process during the step of contacting the glycol ether with a mixed bed of ion exchange resin can include, for example, from 0° C. to 100° C. in one embodiment, from 10° C. to 60° C. in another embodiment, and from 20° C. to 40° C. in still another embodiment. If the temperature is above 100° C., the resin will be damaged; and if the temperature is below 0° C., some of the glycol ethers to be treated may freeze.

The weak-acid cationic ion exchange resin and the weak-base anionic ion exchange resin useful in the present invention can originally contain water (swelled by water in equilibrium condition with water). Water functions as fuel for a hydrolysis reaction to occur under acidic conditions. Thus, in a preferred embodiment, water is removed from the ion exchange resins prior to solvent treatment. In one general embodiment, the content of water in the cationic ion exchange resin and the content of water in the anionic ion exchange resin is decreased to 10 wt % or less, respectively, (i.e., for each resin) prior to use; and to 5 wt % or less in each resin in another embodiment. In one embodiment, a general method to remove water from an ion exchange resin includes, for example, by solvation with a water miscible solvent. In carrying out the above method, a resin is immersed in a water miscible solvent until equilibrium is reached. Then, the resin is again immersed in fresh water miscible solvent. By repeating immersion of resin in water miscible solvent, water removal can be achieved. In another embodiment, a general method of removing water from ion exchange resin includes, for example, by drying the cationic ion exchange resin and the anionic ion exchange resin before contacting the ion exchange resins with an organic solvent. An apparatus of drying and conditions such as temperature, time and pressure for drying ion exchange resins can be selected using techniques known to those of skill in the art. For example, the ion exchange resins can be heated in an oven at a temperature of from 60° C. to 120° C. for a period of time of, for example, from 1 hr to 48 hr under decompressed condition. The content of water can be calculated by comparing the weight of an ion exchange resin before and after heating the resin at 105° C. for 15 hr.

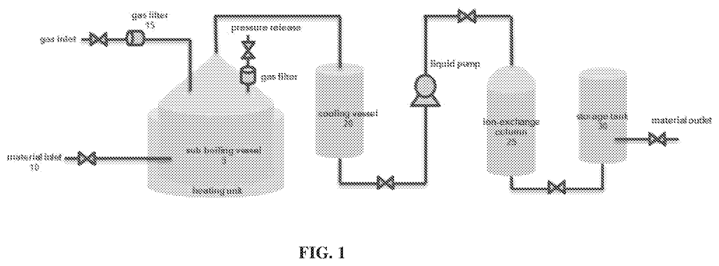

FIG. 1 illustrates a process for purifying glycol ethers according to one embodiment of the present invention. In the embodiment shown in FIG. 1, the process includes a sub-boiling step followed by an ion exchange step. As noted above, in other embodiments, the ion exchange step can be first, followed by the sub-boiling step. Turning to operation of the embodiment shown in FIG. 1, the glycol ether(s) are loaded into the sub-boiling vessel 5 at material inlet 10. The sub-boiling vessel 5 is then filled with inert gas, such as nitrogen and/or argon. The purity of the inert gas is at least 99.999% in some embodiments. To remove particles and dust and keep the inert gas clean, the inert gas passes through gas filter 15. The water content and oxygen content inside the vessel is controlled so that each are less than 20 ppm. The glycol ether in the sub-boiling vessel 5 is heated to a sub-boiling temperature of the glycol ether, wherein the sub-boiling temperature is at least 15° C. less than the normal boiling point of the glycol ether. The pressure in the sub-boiling vessel 5 can be under vacuum or at ambient pressure, in some embodiments. In some embodiments, the pressure can be higher than ambient pressure, for example, due to the gas inlet pressure. The sub-boiling vessel includes a pressure release valve to prevent the accumulation of pressure (e.g., for safety) with a gas filter to prevent particles from entering the air when pressure (gas) is released from the sub-boiling vessel 5. Heating of the glycol ether in the sub-boiling vessel generates vapor which then flows into a cooling vessel 20. In the cooling vessel 20, the vapor condenses into a liquid after cooling naturally. In some embodiments, the temperature of the liquid in the cooling vessel 20 is no more than 90° C. From the cooling vessel 20, the glycol ether can be pumped through an ion exchange column 25 for further reduction of metal content. The ion exchange column is loaded with a mixed bed of ion exchange resins comprising cationic ion exchange resins and anionic ion exchange resins as described above. The flow rate of the glycol ether through the ion exchange column, in some embodiments, is no more than 50 bed volumes per hour. Upon exiting the ion exchange column 25, the purified glycol ether can be stored in a storage tank 30. Sample is collected at a final storage container.

In some embodiments, the whole system including the sub-boiling vessel 5, the cooling vessel 20, the ion exchange column 25, the storage tank 30, and all connecting pipelines are made of SAE 316L grade stainless steel with electroplating, or made of ultrapure perfluoroalkoxy alkane (PFA) or polytetrafluoroethylene (PTFE) polymers. Optionally, in some embodiments, such construction materials can be heatproof materials which can withstand temperatures over 250° C., with inner surfaces coated with ultrapure PFA or PTFE having a coating thickness of at least 2 mm.

In one general embodiment, the targeting metal level of the glycol ether, after the above-described process (sub-boiling and ion exchange), is less than 10 ppb (part per billion) when the feed solvent contains a typical metal level. The obtained glycol ether includes quite low levels of metallic and non-metallic ionic contaminants. The metallic contaminants can include, for example, Li, Na, Mg, K, Ca, Al, Fe, Ni, Zn, Cu, Cr, Mn, Co, Sr, Ag, Cd, Cs, Ba, Pb, and Sn. The concentration of each of these metallic contaminants can be 1 ppb or less in various embodiments. Therefore, the glycol ethers obtained using the process of the present invention can be useful in applications which requires an ultrapure solvent, such as for the manufacture of pharmaceuticals and electronic materials, and especially for use, for example, in semiconductor fabrication processes. High removal rate of metals is necessary to achieve ultrapure solvent. In some embodiments, a process of the present invention advantageously provides more than 80% of metal removal efficiency of the sum of the metals listed above from the glycol ether fed to the process. In some embodiments, a process of the present invention advantageously provides more than 90% of metal removal efficiency of the sum of the metals listed above from the glycol ether fed to the process. In some embodiments, a process of the present invention advantageously provides more than 99% of metal removal efficiency of the sum of the metals listed above from the glycol ether fed to the process.

It is also desired that the purity change of the solvent after ion-exchange treatment is as low as possible as measured by conventional methods such as by GC-FID. For example, in one general embodiment, the purity change of the organic solvents is zero percent (%) or at a level that is lower than the detection limit of a detection instrument (for example, close to zero % such as 0.00001% depending on the selection of the GC detector, selection of the column, and the selection of other measurement conditions). In other embodiments, the purity change of the solvent after ion-exchange treatment is, for example, less than 0.05% in one embodiment; and less than 0.01% in another embodiment.

### Examples

Some embodiments of the present invention are described in detail in the following Examples. However, the following examples are presented to further illustrate the present invention in detail but are not to be construed as limiting the scope of the claims. Unless otherwise indicated, all parts and percentages are by weight.

Various terms and designations used in the Inventive Examples (“IE”) and the Comparative Examples (“CE”) are explained as follows:
“DVB” stands for divinyl benzene.
“MR” stands for macroreticular.
“BV/hr” stands for bed volume/hour(s).

Various raw materials or ingredients used in the Examples are explained as follows:
DOWANOL™ PM is propylene glycol methyl ether (PGME), commercially available from The Dow Chemical Company.
DOWANOL™ PMA is propylene glycol methyl ether acetate (PGMEA), a solvent available from The Dow Chemical Company.
CARBITOL™ Solvent is a diethylene glycol ethyl ether, commercially available from The Dow Chemical Company.
AMBERLITE IRC76 is a weak-acid cationic ion exchange resin commercially available from DuPont. AMBERLITE IRA98 is a weak-base anionic ion exchange resin commercially available from DuPont. Additional details about these ion exchange resins are provided in Table 1:

TABLE 1

| | AMBERLITE IRC76 | | AMBERLITE IRA98 | |
|---|---|---|---|---|
| Type | Weak Acid Cation | | Weak Base Anion | |
| Appearance | Yellow to brown translucent | | Amber translucent | |
| Form | Macro-reticular | | Macro-reticular | |
| Matrix | Polyacrylate | | Styrene-DVB | |
| Functional | Carboxylic acid, —COOH | | Trimethylamine, | |
| group | | | —CH₂N(CH₃)₂ | |
| Ionic | H+ | | Free base | |
| Total | 3.9 | eq/L | 1.35 | eq/L |
| volume | | | | |
| capacity | | | | |
| Moisture | 52-58% | | 54-63% | |
| retention | | | | |
| capacity | | | | |
| Particle size | 675 ± 75 | μm | 590 ± 100 | μm |

For the Inventive Examples, a sub-boiling step is performed first. Inventive Examples 7 and 8 also undergo an ion exchange step as discussed further below. The whole system including the sub-boiling vessel, the cooling vessel, the ion-exchange column, bottles, and connecting pipelines are all made of perflouroalkoxy alkane (PFA) materials.

The vessel for sub-boiling has a volume of four liters. A heating bowl is placed below the sub-boiling vessel to heat the material in vessel. The sub-boiling vessel with heating bowl is placed in a glove box filled with ultrapure argon (assay 99.999%) to control oxygen and moisture to <5 ppm. Particle control is at 100 class clean room level. The pressure is ˜1.5 bar.

Table 2 shows the glycol ethers that are tested as well as the temperature of sub-boiling for the Inventive Examples (IE1-IE8). The sub-boiling temperatures are at least 15° C. less than the normal boiling points of the glycol ethers. The Comparative Examples (CE1-CE4) are not subjected to sub-boiling or ion exchange.

TABLE 2

| | Glycol Ether | Temperature of Sub- |
|---|---|---|
| | (Boiling Point) | Boiling (° C.) |
| Comparative Example 1 | DOWANOL ™ PM | — |
| | (120° C.) | |
| Comparative Example 2 | DOWANOL ™ PMA | — |
| | (146° C.) | |
| Comparative Example 3 | CARBITOL ™ Solvent | — |
| | (196° C.) | |
| Comparative Example 4 | DOWANOL ™ PMA | — |
| Inventive Example 1 | DOWANOL ™ PM | 50 |
| Inventive Example 2 | DOWANOL ™ PM | 70 |
| Inventive Example 3 | DOWANOL ™ PMA | 40 |
| Inventive Example 4 | DOWANOL ™ PMA | 80 |
| Inventive Example 5 | CARBITOL ™ Solvent | 70 |
| Inventive Example 6 | CARBITOL ™ Solvent | 90 |
| Inventive Example 7 | DOWANOL ™ PMA | 80 |
| Inventive Example 8 | DOWANOL ™ PMA | 80 |

For the Inventive Examples, three liters of the specified glycol ether are added to the vessel. The glycol ethers are heated to the sub-boiling temperature specified in Table 2. As a result of the heating, vapor is formed in the sub-boiling vessel and flows out the top of the sub-boiling vessel to a four-liter cooling vessel maintained at a temperature of 20° C. or less. In the cooling vessel, the vapor condenses into a liquid. For Inventive Examples 1-6, samples from the cooling vessel are collected and tested for metal content and water content. The water content is measured in accordance with ASTM E203 using Karl Fischer titration. The concentrations of metals in the solvent samples are analyzed by conventional equipment such as an ICP-MS (inductively Coupled Plasma-mass spectrometry) instrument available from Agilent Technology; and the analytical results are described in the tables which follows herein below. Original metal level (concentration) and metal element ratio are varied by feed solvent lot.

Inventive Examples 7-8 are passed through an ion exchange column as follows. The ion exchange column has a volume of 100 milliliters. 10 milliliters of a mixed bed of ion exchange resins is loaded into the ion exchange column. The mixed bed of ion exchange resins is 50% of a weak acid cation resin (AMBERLITE IRC76) and 50% of a weak base anion resin (AMBERLITE IRA98). The flow rate of the sub-boiled glycol ethers are 10 bed volumes per hour for Inventive Example 7 and 30 bed volumes per hour for Inventive Example 8. After WO 2022/241699 PCT/CN2021/094703 passing through the ion exchange column, the glycol ethers are collected in a sample bottle, and the water content and the metal content are measured as described above.

The water content and metal content measurements are shown in Table 3:

TABLE 3

| Property | CE1 | CE2 | CE3 | CE4 | IE1 | IE2 | IE3 | IE4 | IE5 | IE6 | IE7 | IE8 |
|---|---|---|---|---|---|---|---|---|---|---|---|---|
| Water | 930 | 913 | 975 | 603 | 740 | 447 | 865 | 629 | 858 | 861 | 526 | 501 |
| content | | | | | | | | | | | | |
| (ppm) | | | | | | | | | | | | |
| Sodium | | | 431 | | | | | | 0.25 | 0.19 | | |
| (ppb) | | | | | | | | | | | | |
| Sodium | | | | | | | | | 99.9 | 99.9 | | |
| removal | | | | | | | | | | | | |
| rate (%) | | | | | | | | | | | | |
| Magnesium | | | 2.65 | | | | | | 0.00 | 0.01 | | |
| (ppb) | | | | | | | | | | | | |
| Magnesium | | | | | | | | | 100 | 99.9 | | |
| removal | | | | | | | | | | | | |
| rate (%) | | | | | | | | | | | | |
| Potassium | | | 32.2 | | | | | | 0.28 | 0.16 | | |
| (ppb) | | | | | | | | | | | | |
| Potassium | | | | | | | | | 99.1 | 99.5 | | |
| removal | | | | | | | | | | | | |
| rate (%) | | | | | | | | | | | | |
| Calcium | | | 6.63 | | | | | | 0.03 | 0.22 | | |
| (ppb) | | | | | | | | | | | | |
| Calcium | | | | | | | | | 99.5 | 96.7 | | |
| removal | | | | | | | | | | | | |
| rate (%) | | | | | | | | | | | | |
| Manganese | | | 1.67 | | | | | | 0.00 | 0.00 | | |
| (ppb) | | | | | | | | | | | | |
| Manganese | | | | | | | | | 100 | 100 | | |
| removal | | | | | | | | | | | | |
| rate (%) | | | | | | | | | | | | |
| Iron (ppb) | | | 3.43 | | | | | | 0.07 | 0.11 | | |
| Iron | | | | | | | | | 98.0 | 96.8 | | |
| removal | | | | | | | | | | | | |
| rate (%) | | | | | | | | | | | | |
| Zinc (ppb) | | | 53.0 | | | | | | 0.18 | 0.18 | | |
| Zinc | | | | | | | | | 99.7 | 99.7 | | |
| removal | | | | | | | | | | | | |
| rate (%) | | | | | | | | | | | | |
| Total metal | 2.85 | 3.69 | 532 | 8.95 | 0.29 | 0.32 | 0.72 | 0.62 | 1.09 | 1.13 | 0.65 | 0.61 |
| (ppb) | | | | | | | | | | | | |
| Total metal | | | | | 89.7 | 88.7 | 80.6 | 83.1 | 99.8 | 99.8 | 92.7 | 93.2 |
| removal | | | | | | | | | | | | |
| rate (%) | | | | | | | | | | | | |

The total metal content includes Li, Na, Mg, Al, K, Ca, Cr, Mn, Fe, Co, Ni, Cu, Zn, Sr, Ag, Cd, Sn, Cs, Ba, and Pb.

As shown in Table 3, each of the Inventive Examples contain much less metal and water than the relative Comparative Examples without purification. The metal removal rates of each of the Inventive Examples are over 80%. When the initial metal concentration is at hundreds of ppb level, the metal removal rate could achieve over 99% (see, e.g., Inventive Examples 5 and 6). As shown by Inventive Examples 7 and 8, the sub-boiling step in combination with ion exchange using a mixed bed of cationic and anionic ion exchange resins can remove more metal than using only the sub-boiling step.

1. A process for purifying glycol ethers, the process comprising:
(a) providing a glycol ether to a first vessel, the glycol ether having a normal boiling point at one bar and the glycol ether having the following formula:
R₁—O—(CHR₂CHR₃)O)ₙR₄;
wherein R₁ is an alkyl group having 1 to 9 carbon atoms or a phenyl group; wherein R₂ and R₃ each individually is hydrogen, a methyl group or an ethyl group, provided that when R₃ is a methyl group or an ethyl group, R₂ is hydrogen and provided that when R₂ is a methyl group or an ethyl group, R₃ is hydrogen; wherein R₄ is hydrogen, an alkyl group having 1 to 4 carbon atoms, an acetyl group, or a propionyl group; and wherein n is an integer of 1 to 3;
(b) filling the first vessel with inert gas;
(c) heating the glycol ether in the first vessel to a sub-boiling temperature, wherein the sub-boiling temperature is at least 15° C. less than the normal boiling point;
(d) cooling the vapor from the first vessel in a second vessel to provide a liquid; and
(e) contacting the glycol ether with a mixed bed of ion exchange resins comprising cationic ion exchange resins and anionic ion exchange resins.

2. The process of claim 1, wherein steps (c) and (d) are performed before step (e), and wherein the glycol ether in step (e) is in the liquid from step (d).

3. The process of claim 1, wherein step (e) is performed before steps (a)-(d), and wherein the glycol ether exiting the mixed bed of ion exchange resins is provided to the first vessel in step (a).

4. The process of claim 1, wherein after the process steps are completed, the concentration of Li, Na, Mg, K, Ca, Al, Fe, Ni, Zn, Cu, Cr, Mn, Co, Sr, Ag, Cd, Cs, Ba, Pb, and Sn in the glycol ether are each 1 ppb or less.

5. The process of claim 1, wherein water content and oxygen content in the first vessel are each less than 20 ppm.

6. The process of claim 1, wherein the cationic ion exchange resins are weak-acid cationic ion exchange resins and wherein the anionic ion exchange resins are weak-base anionic ion exchange resins

7. The process of claim 1, wherein the weak-acid cationic ion exchange resin is a macroreticular type resin and the weak-base anionic ion exchange resin is a macroreticular type resin.

8. The process of claim 7, wherein the matrix material of the macroreticular type resins are selected from the group consisting of a crosslinked styrene-divinyl benzene copolymer, a crosslinked acrylic (methacrylic) acid-divinyl benzene copolymer, or a mixture thereof.

9. The process of claim 1, wherein the weak-acid functionality of the weak-acid cationic ion exchange resins is selected from the group consisting of weak-acid carboxylic acid groups, weak-acid phosphonic acid groups, weak-acid phenolic groups, and mixtures thereof.

10. The process of claim 1, wherein the weak-base functionality of the weak-base anionic ion exchange resins is selected from the group consisting of a primary amine group, a secondary amine group, a tertiary amine group, and mixtures thereof.
